(19) United States
(12) Patent Application Publication
(10) Pub. No.: US 2021/0407631 A1
(43) Pub. Date: Dec. 30, 2021

(54) METHODS AND SOFTWARE FOR HISTOCOMPATIBILITY LABORATORY OPERATION AND QUALITY ASSURANCE

(71) Applicants: Marcelo Pando, Georgetown, TX (US); Jorge Moraleda, San Diego, CA (US); Yanying Chen, San Diego, CA (US); MacGregor Belniak, San Diego, CA (US)

(72) Inventors: Marcelo Pando, Georgetown, TX (US); Jorge Moraleda, San Diego, CA (US); Yanying Chen, San Diego, CA (US); MacGregor Belniak, San Diego, CA (US)

(21) Appl. No.: 17/359,748

(22) Filed: Jun. 28, 2021

Related U.S. Application Data
(60) Provisional application No. 63/045,138, filed on Jun. 28, 2020.

(51) Int. Cl. G16H 10/40; G06F 21/31; G06F 16/93; G16H 50/30

(52) U.S. Cl. CPC G16H 10/40; G16H 50/30; G06F 16/93; G06F 21/31

(57) ABSTRACT

A method for histocompatibility laboratory operation and quality assurance whereby software gathers test results from databases. The software creates and assigns tasks to laboratory personnel. The software applies algorithms to perform an antibody quality check and provides the user visualization for reviewing and accepting the test or sending it for rework. The software creates tasks to review approved test results for unacceptable antigens. The software applies algorithms to identify potentially unacceptable antigens and provides the user with an interactive visualization to enable them to decide what antigens are unacceptable. The software uploads unacceptable antigens directly to UNOS. The software gathers data from UNOS about donor offers, compares to patient data, and applies algorithms to help the user perform a Virtual Crossmatch and determine if an offered organ should be accepted. The software generates reports, manages reviews, gathers digital signatures and provides a secure audit trail.

### BACKGROUND OF THE INVENTION

### Field of the Invention

The present invention relates to methods and software to support operation and quality assurance of histocompatibility laboratories that facilitate treatment of organ transplant patients.

### Description of the Prior Art

The success of human organ transplantation depends on the immune compatibility of the transplanted graft organ with the patient. Specifically, histocompatibility means having sufficiently similar antigens/alleles of Human Leukocyte Antigens (HLA) genes. Patients that are candidates for transplantation can develop antibodies to HLA antigens due to sensitization (pregnancies, blood transfusions, etc.). These antibodies to different HLA antigens could, if the donor has any of them, trigger a rejection of the organ thereby jeopardizing the transplanted organ and the life of the transplant patient. To avoid these compatibility issues, the Histocompatibility Laboratory is in charge of typing and matching patients with potential donors and monitoring the immune status post-transplant for rejection. Histocompatibility laboratories also perform clinical or research testing that requires strict quality controls.

In the HLA lab, patients are tested for the presence of antibodies to HLA antigens that could trigger a rejection of the transplanted organ. Accurate timely analysis and review of the results of the test is essential to guarantee proper patient care. The turnaround time (TAT) of this test, depending on the laboratory, could be from 48 hr TAT for a STAT test (statum; Latin for immediately), to 15 days for a screening test. The test is separated into two parts, the bench work and the analysis work. The bench work can take 4-8 hours depending on the number of samples to be tested, but the analysis work can take several days depending on the number of patient samples and the complexity of the analysis and review.

The histocompatibility laboratories have a quality assurance program to monitor and assure the quality of work performed prior to the testing (pre-analytical), during the testing (analytical), and after the testing (post-analytical). Personnel have to be competent to run each test, testing methods must be validated, and all kits and reagents have to be tested before running patient tests to assure quality (quality control). The quality assurance of the laboratory must be documented and properly tracked to comply with federal and state regulations (CLIA, FDA, etc). Laboratories struggle to document, track, schedule, and organize records to properly comply with regulatory agencies and guarantee the quality of the laboratory operations. Some laboratories use generic computer programs such as spreadsheets and database tools that do not optimally follow the quality processes of the laboratory. However, most laboratories use paper records that are cumbersome to maintain, file, and use in the laboratory workflow and audit processes. Furthermore, laboratories analyze and review the testing results manually without clear algorithms. Manual review requires many cycles of checking and double-checking to avoid mistakes. Good review systems reduce test result mistakes, but at a high cost of personnel time. There are no computer programs that specifically target the critical workflows and quality management of a histocompatibility laboratory.

As part of pre-analytical quality assurance, all test kits and reagents must undergo rigorous quality control testing that must be documented. These quality control records are reviewed and approved prior to any patient testing. The paper trail for pre-analytical quality assurance is usually saved in paper folders that are organized on shelves. Records must be kept for years to comply with local and federal regulations. Audits are cumbersome due to the use and management of paper records.

The workflows associated with a kidney transplant are representative of the workflows that are typical for other organ types in histocompatibility laboratories today. For the sake of brevity, however, the prior art is described using a kidney transplant example. A patient must be evaluated by the transplant team before being approved to be added to the waiting list for a transplant. As part of the evaluation process, blood is drawn from the patient and sent to the histocompatibility laboratory. The histocompatibility laboratory receives the blood and processes it to obtain serum for antibody testing and to obtain DNA for genetic typing of HLAs. Many tests exist for accurately determining the patient's antibodies and HLA specificities. Tests such as SSOP (sequence specific oligonucleotide probes), SSP (sequence specific primers), Sanger sequencing, NGS (next generation sequencing), can be used alone or in combination. Similarly, many tests and combinations of tests exist for antibody determination such as Elisa, CDC, single antigen beads, phenotype beads, and flow cytometry PRA beads. The end result of the test or tests is data about the HLA-A, B, Cw, DRB1, DRB345, DQA1, DQB1, DPA1, DPB1 antigens and, if needed, the HLA allele for each antigen. The data is normally stored in a laboratory database such as Microsoft SQL Server. The laboratory must first review the results against controls, and compare to prior results, if any, to determine if the results can be trusted or if there are signs of a physical problem during the testing such as contamination of a sample from an adjacent sample. This review is called an Antibody Quality Check. The process must be documented and reviewed by senior technicians and/or the laboratory director before the laboratory proceeds to use the data to determine the patient's antibodies to HLA.

Ideally, once approved, the results from a new patient sample would immediately be reviewed to determine if the patient's antibodies to HLA have changed and those changes would immediately be communicated to UNOS to ensure matching based on the most current data. Unfortunately, it is common for new results to pile up for review in regular batches, typically weekly. Each week, a technician works through printouts or queries a database to identify new and approved results that need to be analyzed for unacceptable antigens. The laboratory team must then perform an Unacceptable Antigen Analysis to evaluate the data to decide if the patient is sensitized and determine what antibodies to HLAs should be avoided. This determination is also manual and painstaking, because the technician must either print the data onto paper for review or export it for review in generic software programs such as spreadsheets. After the technician makes an initial determination, the data and analysis must be reviewed by senior technicians and/or the laboratory director. Sometimes the results are suspicious and tests and/or analysis must be repeated until a reasonable result is approved. The determinations are often subjective and practices vary widely across HLA laboratories at different transplant centers. Once approved, the laboratory must post the results to the UNOS system so that the patient can be matched with potential donors. The information sent to UNOS must be double-checked to ensure that no clerical errors have been made. A complete document trail of the analysis must be stored; typically using large volumes of paper records and a filing system.

Unfortunately, processes such as infection and blood transfusions cause the patient antibodies to HLA to change over time. transplant centers, therefore, regularly repeat the entire analysis to ensure that UNOS has up to date information about the patient to enable finding the best possible matches. Patients are tested regularly, typically each month, to determine if their antibodies to HLA have changed or not. It is standard to manually compare previous samples to the new samples to determine if new antibodies are developed. The comparison process is usually poorly documented, if documented at all.

When a donor becomes available a histocompatibility laboratory performs HLA typing on the donor and uploads the results to UNOS. UNOS applies algorithms to match the donor with patients that are registered for transplantation. UNOS priorities the patients and notifies transplant centers of the potential match. Many transplant centers then perform a detailed immunological assessment of the patient-donor pairing to determine if the offered organ will be accepted or declined. This assessment is called a Virtual Crossmatch. The methods for performing the Virtual Crossmatch are ad hoc, manual, and not standardized across transplant centers. The process may start by logging into UNOS and accessing information about the HLA typing of the donor. The information may be printed or inserted into generic software tools like spreadsheets to compare the HLA typing of the donor with the HLA typing of the patient. The manual process is prone to inaccuracies, and time limited, because the organs are only viable for a limited time after the donor dies. The review may be done merely by a technician or sometimes reviewed by supervisor or director, if time allows. In many transplant centers, the organ is accepted or rejected based on the Virtual Crossmatch. The laboratory will also perform a physical crossmatch test that involves isolating lymphocytes from the donor's blood and mixing it with sera from the patient to identify potential incompatibilities. Time is of the essence, because the longer the organ waits the lower the odds of successful transplantation. Ideally, the laboratory should document the entire Virtual Crossmatch and review process however, there is often not time for this and an organ is accepted by verbal communication by phone, text message, or email. After a transplant, the histocompatibility laboratory continues to monitor the patient for signs of rejection that can be treated more successfully if caught early.

Antibody Quality Check

Different technologies may be used to determine the HLA specificities to which the patient has antibodies. The most common technology used in the US and across the globe, is based on the Luminex technology. Luminex uses a specialized flow cytometer designed to analyze beads that are loaded with HLA antigens. The beads are labeled with two different fluorochromes (FL1 and FL2) to categorize groups of beads with similar features and are able to run all HLA categories all at once, see FIG. 1. Each group of beads is loaded with one particular HLA antigen or a few antigens. More than 90 different HLA specificities are analyzed in one test by using many groups of beads. Two tests are usually performed to analyze HLA Class I and Class II antigens. For each test, patient serum is incubated with beads. If any HLA antibody is present in the patient serum, the antibodies will bind to the specific group of beads with the HLA antigen. The beads are then washed several times. Then, a secondary antibody labeled with Phycoerythrin is used to detect the antibodies bound to HLA antigens. The beads are washed again. The fluid for each patient is then added to a tray for analysis. Today, trays have up to 96 wells that can be used to analyze up to 94 patient samples (plus 2 control samples) in a single run. The tray is loaded into the flow cytometer system (typically Luminex). The system works with one well at a time and passes the fluid through the flow cytometer which uses lasers to count the beads that are reactive and thereby measure the presence of antibodies to specific HLA antigens. The flow cytometer automatically saves the information in a database such as SQL Server. A technician must gather the data from the database into a spreadsheet or printout and then compute several quality metrics, analyze the metrics and ensure that the metrics pass muster.

The technician first reviews data from a negative control well and positive control well to determine if they are in acceptable ranges. If the global controls are within normal limits, the technician proceeds to analyze one well at a time. For each well, the technician first examines the data from internal negative and positive control beads in the well. If the controls are in the acceptable range, the technician must then gather historical data about the patient being analized in the well to compare the current results against past results. This is a highly manual process that requires ad hoc queries to the database and collating results on paper or in spreadsheets. The technician painstakingly copies and pastes information, yet errors still are inevitable in the collation process. The technician looks at changes in each antibody profile across the current result and one or multiple prior results to determine if there is a change in antibody profile that indicates a new sensitization or an error such as a problem with sample integrity or a sample being switched accidentally.

The technician may order rework if there are problems or document and pass along the results for further review and approval or rejection by supervisors and/or laboratory directors depending on the processes in place at the particular HLA laboratory. Different approaches are currently used in laboratories to follow up on tests that are not acceptable or are questionable. Laboratories often have a repeat test list on a paper, sticky notes, in a spreadsheet, or via email. Sometimes, there is no written record and the repeat is requested by a reviewer verbally. These approaches are not practical and many times the need for followup testing is neglected.

Once the quality of each sample is verified, it is marked as ready for further examination to determine if new antibodies exist and if changes to the patient's UNOS record must be made to ensure the best match based on the latest data. The entire process must be documented for lab audits which consumes more valuable time of the laboratory team. Many laboratories simply keep paper records, while others organize and store spreadsheets.

Unacceptable Antigen Analysis

On a regular basis, typically weekly, a technician will search for new results from the plates that have been run and samples that have passed quality assurance. The search may be done via paper records, spreadsheets, or ad hoc SQL queries of the data produced by the flow cytometry machines. The new results must be analyzed to determine if the patient has developed new antibodies and identify donor antigens that pose an unacceptable risk of organ rejection. The technician, however, is overwhelmed with data. Each patient is tested for more than 200 different HLA specificities that can be added to the list of unacceptable antigens in the UNOS matching system. The technician must run ad hoc queries to gather the data, typically paste the data into a spreadsheet, manipulate the data with sorting and filtering, and then use rules-of-thumb and experience to identify unacceptable antigens. Some or all of the HLA specificities of antibodies detected by the test will eventually be listed as unacceptable antigens in the UNOS database. The determination of what to report to UNOS is a complex decision that depends, in part, on the magnitude of the measurement from the test, but also on many other factors. For example, the distinctive reactivity of each patient's serum is called the patient's HLA antibody profile. HLA antigens can be grouped by similarity at the protein and epitope level. These features present another level of complexity in the analysis. A patient's antibody profile can be defined by antibody groups with the same epitope. Some reviewers may decide to list unacceptable antigens that are not reactive but are part of the antigen group. Also, a patient may have some unacceptable antigens listed that were positive on prior tests, but currently negative (historical antigens). The historical antigens may or may not be registered with UNOS depending on the particular approach of the laboratory and transplant center. Therefore, the analysis of the patient's HLA antibody profile is not just a determination of what is positive and negative, but an intricate analysis of many factors and tests that altogether will determine how likely the patient will receive a transplant. This complex and subjective analysis can also be influenced by the level of risk that the transplant center is willing to take and the beliefs of the laboratory director in determining what is prudent. The complexity and subjectivity of the analysis is highly dependent on human judgement in conjunction with the accuracy and analysis of the test results. This can lead to inconsistent or erroneous results that compromise patient care.

Regardless, once the list of unacceptable antigens is determined the laboratory team then records the unacceptable antigens, compares them with what is currently listed in UNOS and then passes all of the information on to a supervisor and/or laboratory director for review and approval. The technician must keep track of any orders for rework and ensure that documents are filed electronically or more typically, in paper files for periodic laboratory audits. The list of unacceptable antigens is then communicated to the UNOS.

Once the unacceptable antigens are approved and documented in the review, they must be accurately uploaded to UNet (UNOS informatic system). Unfortunately, the international HLA naming convention, defined by W.H.O., is slightly different from that used by UNOS, which forces the laboratory team to manually translate the results into the appropriate format for communication to UNOS. Laboratories communicate unacceptable antigens to UNOS in two primary ways. One, they log into the UNet website, access the patient transplant registration, and use a web page with checkboxes to select the unacceptable antigens. A sensitized patient may have from one to over a hundred antigens to list and review every time a new sample is tested, a very time consuming process. Two, they log into UNet and upload a comma separated values (CSV) file in an agreed upon format that specifies the unacceptable antigens for a particular group of patients. With either process, there is grave risk to the patient if any clerical errors are made when copying the unacceptable antigens from the laboratory records to UNOS. After the upload, another technician must perform a double-check, consuming substantial time, to ensure that the unacceptable antigens recorded for a particular patient are correct. The entire process must be documented for review in periodic audits. This is often done by printing results and storing them in paper folders or scanning paper documents into PDF files and saving them in a computer file system. Systems must be backed up to avoid losses due to computer failures. Once the antigens are updated in UNet, UNOS adjusts the organ matching algorithm accordingly to eliminate organs that would be incompatible with the patient's current antigen list. The entire unacceptable antigen analysis must be repeated periodically, normally monthly, for each patient.

Virtual Crossmatch

When a deceased donor becomes available, a histocompatibility laboratory performs HLA typing on the donor. The results of the typing are communicated to UNOS. UNOS performs a match run to identify the list of patients that are the highest priority for receiving an organ from the donor. Each transplant center is notified if any of their active and listed patients receive at least one offer from UNOS. The transplant center must rapidly and accurately decide if each offer is acceptable under their criteria and accept or reject the offer. The criteria may consider, but is not limited to, the patient's previous organ transplants, HLA matching, the patient's antibody history, etc. Many transplant centers perform a Virtual Crossmatch to help determine if further testing should be performed on the organ or if the offer should be immediately accepted or declined. Often, a transplant center accepts an organ only based on the Virtual Crossmatch assessment and performs a retrospective physical crossmatch to confirm the results of the Virtual Crossmatch. If the results of the physical crossmatch prove the Virtual Crossmatch incorrect, the organ may, unfortunately, be wasted. Therefore, the importance of an accurate and reliable Virtual Crossmatch is crucial.

Most laboratories perform Virtual Crossmatch manually, while others use rudimentary software tools that only consider single antigen beads measured on the patient and the HLA typing of the donor. First the technician obtains the HLA typing of the donor by accessing UNet and downloading the donor HLA typing data. The technician then gathers the latest data about the patient from previous unacceptable antigen analysis via printouts, spreadsheets, or ad hoc SQL queries. The technician then uses spreadsheets or rudimentary tools mentioned above to compare the HLA typing of the donor to the HLA typing of the patient to determine compatibility across HLA-A, B, C, DRB1, DRB345, DQA1, DQB1, DPA1 and DPB1. For each mismatched HLA antigen or allele, the technician searches for HLA antibodies, over the patient's history of HLA antibody testing, that could be reactive with the donor. Rudimentary software tools only assess some differences that are present and require additional time consuming manual analysis to ensure a successful Virtual Crossmatch.

Sometimes the laboratory also performs a physical crossmatch by acquiring a sample from the donor, performing the required HLA typing tests and then comparing against the latest patient results. Each laboratory usually has one technologist on call at every moment of every day to ensure cross matching can be done as fast as manually possible. This manual Virtual Crossmatch process is time limited, and is prone to many inaccuracies. Depending on the workflow of the laboratory, this Virtual Crossmatch may be done only by a technologist, or optionally reviewed by a second technologist, supervisor, or director. In many transplant centers, this assessment not only will determine the acceptance or decline of the organ for transplantation, but also if the transplant can be performed before running the physical crossmatch test. Laboratories report the results of the Virtual Crossmatch in many different ways. Most commonly, a verbal communication on the phone or a written phone text or email. Unfortunately, laboratories rarely have time or resources to provide a well documented report with the results, signature of the technician, reviewers, and approvers.

### Definition

Histocompatibility laboratory: any laboratory that performs analysis and testing of HLA genes, other non-HLA related genes (MIC, KIR, Endothelial antigens, HP antigens, tec.) implicated in solid organ, bone marrow and stem cell transplantation, transfusion compatibility, and any other related tissue compatibility. Included are laboratories that test HLA genes or antigens for HLA associated diseases (autoimmune, cancer, disease association, pharmacogenetics, population genetics, research studies, etc.).

Part 11 Compliance: (FDA) Title 21 CFR Part 11 regulates data security and trustworthiness of computer systems used for document storage in the medical industry.

Plate or tray: laboratory plasticware with multiple tubes or wells setup in the same unit. Usually the plate has 12 columns by 8 rows of wells and simplifies testing of multiple samples from multiple patients at once.

Similarity Score (SC): algorithm used to compare the current sample run with previous samples. The algorithm detects general variations in the pattern of positive reactions and also single variations.

Phycoerythrin (PE): fluorescent protein used to label antibodies and perform flow cytometric analysis. It is excited by a 488 nm Laser and emits 576 nm.

Mean Fluorescence Intensity (MFI): Value that relates to the strength of reactivity of an antibody to the specific antigen. Here, MFI may refer to a raw value or a normalized value.

ETag: The entity tag is part of HTTP, the protocol for the World Wide Web. ETags can be used for optimistic concurrency control to prevent simultaneous updates of a resource from overwriting each other.

### Statement of the Objectives

The overarching objectives of the invention are to: improve the quality of organ transplant patient care, improve the quality of histocompatibility laboratory operations, promote sharing of best practices for histocompatibility laboratories, reduce the amount of time spent on laboratory paperwork, accelerate the completion of time-sensitive laboratory tests, and streamline periodic laboratory audits.

### SUMMARY OF THE INVENTION

The Summary of the Invention is not intended to limit the scope of the invention, but rather aims to facilitate a basic understanding of the invention described in The Detailed Description of the Preferred Embodiment.

In one form of the invention, a suite of computer software programs work together to improve the quality assurance process of histocompatibility laboratories. The suite has three main software programs, the Antibody Quality Check Process, the Unacceptable Antigen Process, and the Virtual Crossmatch Process. The three main programs share a user interface that enables the user to start processes, see a list of tasks that they are qualified to complete, complete those tasks, and search and audit their past work.

The Antibody Quality Check Process can be automatically or manually triggered when new tests have been completed in the laboratory. In either case, when an Antibody Quality Check Process is triggered, the software adds a task to a list and assigns it to be completed by a list of qualified users. The software lets the user pick up the task for work. When the user starts working on the task, the Antibody Quality Check Process gatherers data from databases that contain test results. The software synthesizes the test results, applies algorithms to automatically detect quality problems, and generates a visualization of the physical plate that was used in the test along with overlays that identify issues for further examination. The software enables the user to drill into a detailed view of each well on the plate, visualize data from the well, compare against previous samples, add comments about their analysis, and either send the sample for retesting or generate a report, sign the report, and send it along for further review by supervisors or laboratory directors. Digital signatures ensure the integrity and auditability of the entire review process. Once antibody test results have been approved and verified, the software automatically triggers a sequential analysis including an Unacceptable Antigen Process, for each patient with a new valid result.

The Unacceptable Antigen Process can be automatically triggered by the completion of an Antibody Quality Check or manually started by searching for recently completed tests. As with the Antibody Quality Check Process, a task is created and added to a list where qualified users can see and start working on the task. The Unacceptable Antigen Process gathers data from databases that contain the test results. The software synthesizes the test results and applies algorithms that execute editable policy rules for each organ type and antigen to automatically identify potentially unacceptable antigens. The software provides the user with a sortable and filterable list of antigens where potentially unacceptable antigens are highlighted. The software enables the user to examine the data and select which unacceptable antigens to report to UNOS. The software enables the user to authenticate to the UNOS website and download the currently registered unacceptable antigens for comparison to the newly proposed unacceptable antigens. The user can either revise their selections or directly upload the new unacceptable antigen list to UNOS. UNOS then uses the results to provide the best potential donor matches to patients. Every decision is recorded by the software for later audits.

When organs become available for transplant, the tasks in the Virtual Crossmatch Process can be triggered automatically or manually. The software provides qualified users a Virtual Crossmatch task that manages the workflow of verifying the compatibility between the donor and patient. The software gathers UNOS authentication information from the user, connects to UNOS, and downloads data about the donor. The software connects to databases and gathers similar information about the patient. The software applies algorithms to automatically compare the donor and patient profiles and identify potential issues. The software then provides the user with an innovative visualization to allow the user to compare the patient and donor profiles to determine if the organ offer can be accepted or must be declined. The entire process is automatically documented for future audits.

The software provides a user interface for searching and visualizing completed tasks to accelerate reviews and audits. Every process step of every completed task is searchable. For each matching step, the user can drill in to see exactly what the user saw when they were working on and completing the step along with any comments they made. The data is immutable and is linked with chains of digital signatures to ensure the integrity of the audit process.

### BRIEF DESCRIPTION OF THE DRAWINGS

FIG. 1 is a plot showing the bead categorization for the HLA Class I single antigen bead test. Each group of beads are loaded with one HLA specificity (shown A2 and B7).

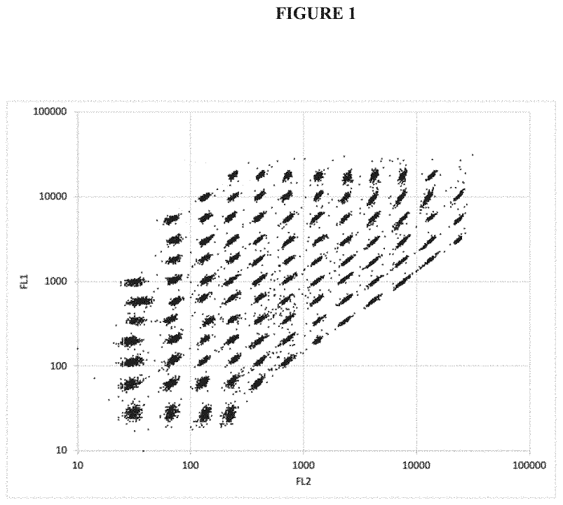

FIG. 2 is a flow diagram of a process for Antibody Quality Check.

FIG. 3 is a flow diagram of a process for determining Unacceptable Antigens. The process includes steps for locating test results, selecting results to analyze, analyzing results, and uploading results to UNOS.

FIG. 4 is a diagram that illustrates a visualization for comparison of old and newly proposed unacceptable antigens.

FIG. 5 is a flow diagram of a process for performing a Virtual Crossmatch and recording the results.

FIG. 6 is a visualization of donor typing from UNOS and patient typing for multiple patients. By clicking in each Patient's MRN Field, the software highlights the mismatched antigens for each Locus in the donor's row. In this case, the patient MRN 1111111 is selected, shown in light gray, and mismatched antigens in dark grey.

FIG. 7 is another visualization of donor typing from UNOS and patient typing for multiple patients. In this example, MRN 2222222 is selected, software highlights the patient's row in light gray, and the donor typing mismatches in dark grey.

FIG. 8 is a detailed view of the match between the donor and patient. In this example, all the antibody data for patient MRN 1111111 is shown. The software automatically shows the MFI DSAs that are highly significant in the table. Patient MRN 1111111 has DSAs to HLA-A30, B44 and DR15 of the donor, automatically highlighted in dark grey. The second table shows the MFIs for each DSA across sample dates to facilitate longitudinal comparison. The view includes a button to display a line plot of the DSA's by date.

FIG. 9 is a graphical view depicting a line plot of the DSA MFIs for each of the 3 DSAs detected in patient MRN 1111111 against the donor.

### DETAILED DESCRIPTION OF THE PREFERRED EMBODIMENT

### User Authentication, Authorization, and Document Authenticity

A critical aspect of quality control is to be able produce an audit trail that specifies exactly who did what and when. In one form of the invention, the software automatically records what is being done, and who is the currently authenticated user, according to some authentication method. In one embodiment, this can be done using a logging and a password, in another embodiment, this can be done taking some biometric measures unique to the user, in yet another embodiment, authentication is performed by demonstrating possession of a trusted object (such as receiving a text message in a given phone that only a given user may have). However in all these cases, there is an initial configuration step in which the authenticating data (password, biometric database, or cellphone number database) must be associated with a human being. An error accidental or deliberate in this step will result in a compromised system when someone may authenticate themselves with a false name. Thus during initial user creation, when authentication data is associated with a user id, including name and email address, the system in addition establishes a security chain of trust when the person creating the new user (who must also be a previously authenticated themselves) provides confirmation that they have personally validated that the authenticating data indeed belongs to the intended individual. This chain of trust is kept in the server via cryptographically secure digital certificates. In particular, the creating user issues a digital certificate to the created user signed with the creating user private key. It is important to note that the chain in a particular deployment starts with the user that performs the installation and who uploads their personal digital certificate to the server, to whom only they know the password and that has been issued to them by their supervisor. Thus for each end user there is a verifiable chain of trust going all the way to the root certificate of the software vendor, whose public key is prominently displayed publicly in various locations, thus avoiding the potential for forgery.

In addition to providing verification of the user's identity, the digital certificates issued to each user and secured by their password, also provide a mechanism for digitally signing documents, a critical aspect of quality assurance.

When a new user is created by an existing user, the newly created user is issued a set of privileges that control what actions the server will allow them to perform. This is important for privacy and quality control issues, as it guarantees that only users that have been authorized to perform certain procedures and checks can perform them, as well as ensuring that access to confidential information is only performed by authorized users.

Individual actions are protected by fine-grained privileges. In order to simplify assignment of users to privileges, in one embodiment, the system allows the creation of “roles” which group privileges, so that users can be assigned to roles and be granted the privileges associated with the role. It is useful to remark that while the set of privileges is determined by the requirements of HLA laboratories, and thus common to multiple transplant centers, the set of roles is specified at each transplant center in ways appropriate and specific to the transplant center.

### Signature Sequence Configuration

Many laboratory processes require a series of signatures and reviews. In one form of the invention, the software provides an interface for defining review and signature workflows. In particular, for every report type that the system provides, an authorized user specifies the sequence of roles that need to review and digitally the report once it is produced. This is important because not all reports required the same level of verification.

What this means is that the specification for a category of reports is provided in terms of a sequence of roles, while for any particular report a sequence of individuals (each belonging to the appropriate role) will actually perform the review and signature. The system ensures that at every step of the sequence only users having the required role are shown the report, and are allowed to sign it. Once this happens, the digital signature is added to the document, along with a datetime stamp, and users having the next role in the sequence are shown the report (including the signatures up to that point) for further review.

### Database Connection Configuration

In one form of the invention, the user must configure a connection to the database that contains the patient data for analysis. The user is presented with a list of processes. The user selects the Configure Connection process, and if authorized for that process, can start a new instance of the process. The user is presented with a list of information that is required for making a connection, including: the server URL, database name, database user ID, database password, database driver name, if the connection must be encrypted, and if there is a Trust Server Certificate. The software provides a button that enables the user to test the connection parameters and either verify a successful connection or diagnose erroneous entries by reading understandable error messages. Once a connection is successfully established, the user can click on a Save button that tells the software to remember the connection settings for use by all of the processes contained in the server. As with everything else, the server preserves a record on who performed this configuration and when.

### Report Header Configuration

In one form of the invention, the software enables the user to customize the header that will be displayed on all laboratory reports. The software includes a template with suggested information including but not limited to, the laboratory directors first name, last name, and credentials, the name of the laboratory, the hospital system associated with the laboratory, the physical address of the laboratory and the laboratories CLIA identification number. In one embodiment, the software provides a high-level user interface that enables the user to fill in this information and produces a LaTeX source file that eventually is rendered as a report header. In addition, the software allows full customization of the header via a low-level LaTeX editor for arbitrary modifications and customization of the formatting of the report header.

The user interface has a button to reset the header back to the default template. The user interface has a button to reset the editor to the previously saved state. The editor also has a button to save the current header template and exit the editor.

### Antibody Quality Check Process

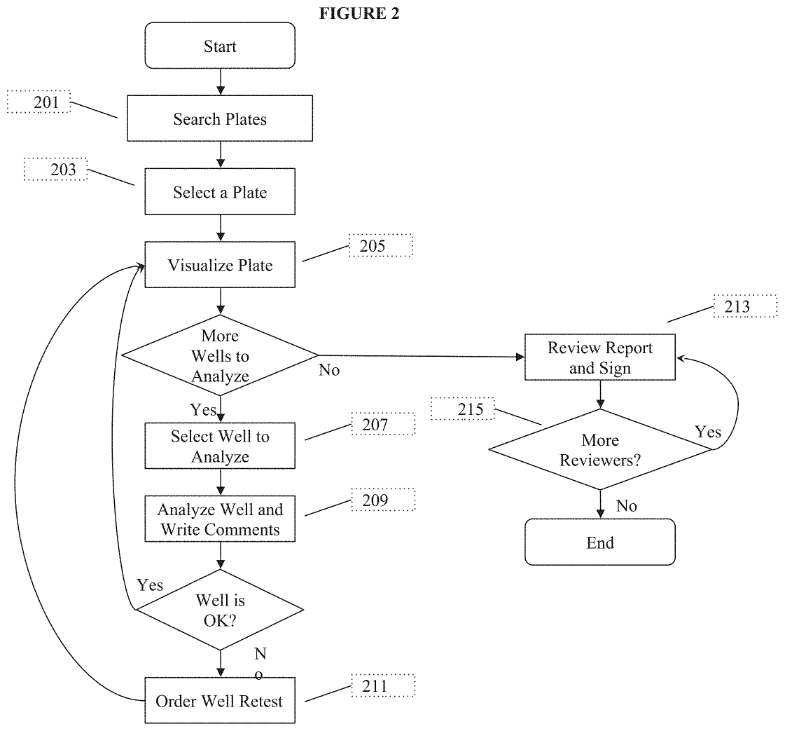

A process flow diagram of one embodiment of the Antibody Quality Check process is illustrated in FIG. 2. In one form of the invention, the Antibody Quality Check process starts at the time the tray (plate) antibody test run is finished and imported into the Fusion or other similar software that stores the results in a database. Data about the plate run is automatically transferred to SQL Server or another database. The software regularly monitors the database for new plate run results. In one embodiment, a BPMN cyclic process running on a timer periodically checks a laboratory database for the latest plate run results and compares them with the latest appropriate Antibody Quality Check results stored in the softwares database. When plates appear in the laboratory database with dates more recent than the last processed plate, those plates are scheduled for review and check. The last scheduled plate is stored in the server database so in the next cycle the software will only search for plates more recent than this one. When data from a new plate is available in the database, the software automatically creates a pending task to review that plate and adds it to a list of pending tasks that need attention from the user. The software may notify users of pending tasks by methods such as, but not limited to, text message, email, or a phone call. This ensures that users are immediately aware that new data is available and needs to be reviewed, thereby providing a central location to track to-do items, replacing ad hoc tracking methods such as post-it-notes, and reducing the time to get vital information back to the patient care team and UNOS.

In one embodiment, the software shows, for each pending task, the type of the task, the date and time when the task was started, the process of which the task is a member, the date and time when the process started, and an instance name that includes the date and identifier of the plate being analized. The software lets the user sort the table of pending tasks by any column to identify tasks by task name, task start time, process name, process start datetime, instance name, or other metrics. The software shows the user all tasks that are pending, even tasks that the user may not have permission to complete. The software permits a user to reassign tasks if they have sufficient privileges. The software has a button that lets the user bring the tasks that they are able to complete to the top of the list. The software also has a button to process the next task in the queue that can be completed by the user given their permissions. In addition, the software allows the user to save their work and resume it at a later time.

In another embodiment, an Antibody Quality Check can also be started manually. The software provides a list of processes that can be started by the user. The software enables the user to start a new instance of the Antibody Quality Check process. The first step of the Antibody Quality Check is to search for plates that need analysis 201. The software lets the user select a range of dates that will be used to identify plates for analysis. When the user selects the date range, the software automatically attempts to connect to the database that contains the results from plate run results. If the connection fails, the software provides a meaningful error message to the user. If the connection succeeds, the software shows how many plates were found in the laboratory database in the specified date range and populates a menu that lets the user select which plate they wish to check. The software has a Check Plate button that triggers the software to load all data about the selected plate from the database. The software then automatically identifies all patients on the current plate and queries the database to identify all past plates where data about each patient is available. The software then compiles a complete history of the plate data for each patient and proceeds to the next step.

Next, the software presents the user with a visual representation of the plate to be analyzed 203. The software provides a view that shows information related to each position on the plate such as the internal negative and positive control, the date of the current sample and date of previous samples, if there is any specificity with low bead counts, and similarity score that gives the reviewer an idea of similarity of results compared to the previous sample. The software shows the identifier of the plate, the date of the plate run and a catalog ID of the plate. The catalog ID is important for setting the context of the analysis, because there are different types of plates that can be used for analysis, each with their own variations on the antigens that can be detected. The software also shows a textual representation of the location of the positive and negative control wells, the total number of patient wells, and the number of patient wells for which there is data available from previous plates.

The software provides a tabular representation of the plate where each cell in the table represents a well on the plate. The software shows the rows of the plate, A through H, and the columns of the plate, 1 through 12. The software also provides a text entry field for making and saving comments about the plate. Each cell is color coded so that the user can easily identify if a cell is the negative control well, the positive control well, if a well has no previous samples associated with it, if a well has previous samples that were similar, if a well is out of the normal range, has a problem such as a bead issue, data that is dissimilar, or data that is very dissimilar from what was found in previous plate runs. The software shows a legend to help the user understand the colors.

Multiple statistics are used to compare wells and ensure the quality of the data in one well. In one embodiment these statistics include:
The MFI of the internal negative control (a bead that is not supposed to react with any substance and thus should always have a low MFI). A value outside a pre-established range is flagged.
The MFI of the internal positive control (a bead that reacts with the secondary antibody giving a determined MFI.). A value outside a pre-established range is automatically flagged.
The deviation of the external negative control serum beads MFI with respect to statistical values of the negative control beads in previous plate runs. A statistically significant deviation is flagged.
The deviation of the external positive control serum beads MFI with respect to statistical values of the positive control beads in previous plate runs. A statistically significant deviation is automatically flagged.
The number of beads of the external negative control well where the MFI exceeds a certain value (An external negative control well should have a non reactive (Negative) serum in it and thus all beads should show low MFI's except for the well's internal positive control). An external negative control well with a statistically significant number of beads showing a high MFI is automatically flagged.
The number of beads of the external positive control well where the MFI is below a certain value. An external positive control well contains a pool of positive serum samples that should react to most beads. Thus an alternative embodiment compares the distribution of values of the positive control with a statistical distribution of previous values of the same bead from previous plates in addition or instead just looking at the number of beads with relatively low values. An external positive control well with a statistically significant deviation relative to previous positive control wells is automatically flagged.
In one embodiment, the software compares the bead counts against thresholds to determine if there are enough beads of each specificity to provide a statistically significant measurement of MFI. For example, if the count for any specificity is below 50 a supervisor must review the result and if the count is below 40 the test must be repeated. In another embodiment, the software compares the distribution of bead counts of each specificity when compared to statistical distributions of previous samples. In general, there should be enough beads of each specificity to provide statistically significant measurements of MFI. Through sampling randomness it is not uncommon to have some beads below this threshold. This is automatically flagged. Moreover when the number of beads with low values deviates in a statistically significant manner from what would be expected from random sampling, this is automatically flagged.
The ratios of each bead MFI with respect to the value of the same bead MFI of previous samples of the same patient. Any beads that show ratios that deviate significantly from one are automatically flagged so they can be investigated and explained, since the default is to expect that a patient's antibody profile does not change over time without reason.

The software reduces the likelihood of errors by automatically identifying wells where at least one of the above quality checks provides suspicious results using a score. This score indicates samples that should be reviewed and helps identify issues such as: the sample was compromised, the sample switched with another, new donor specific antibodies are present, new sensitization is present, and/or new antibodies are present. Unlike representations used in the field today, the tabular representation allows rapid identification of problems with the plate that are only apparent when the visualization supports identifying spatial correlations, such as a splash during the bench work process where several contiguous wells show differences with previous samples.

The software shows information in each cell from the associated plate well. The information includes, the patient first name, patient last name, patient medical record number, the lowest bead count for a specificity, the strength (MFI) of the internal negative and positive control, the date of the current sample, the date of the previous sample, the similarity index, the number of beads below a certain MFI threshold, the number of beads that deviate significantly in the strength (MFI) from the previous sample of the same patient, a button to trigger examination of the well details, and a status indicator that shows if a well has been approved or sent for retesting. The technician uses this information to identify samples that need repeats.

In one embodiment, the details about each well can be reviewed in any order by selecting a well to analyze 207. The technician usually starts by reviewing the details for the negative control well. The software shows the location of the negative control well, labels the negative control well, and color codes the negative control well. Statistics are included in the negative control well visualization to enable the technician to verify that the negative control values are acceptable.

Next, the technician typically reviews the details for the positive control well. Including how many beads are below a certain MFI threshold and how many deviate significantly from previous measurements. If there was a low bead count in the positive control sample, the user can investigate further by clicking on the well to open a detailed view. In the detailed view, the software shows general information such as the tray identification string, the date of the tray and the catalog ID. The software also shows the MFI values for the control well's internal negative and positive control beads. The software shows the number of low count and low value beads found in the well. The software provides an automatic comparison that identifies the number of high variance beads in relation to prior positive control runs, the number of plates in the comparison, date of the earliest plate and the date of the most recent prior plate used in the comparison. The software also provides an area for the technician to enter and save comments about their observations of the positive control well. Finally, the detailed view of the positive control well shows a box plot 209 of the current vs previous bead intensities for the positive control for each type of bead in the well. The box plot helps the user understand the normal range of the MFI for each bead. For each bead type, the software shows a dot that marks the MFI of the current positive control measurements in relation to the box plot of historical values. The MFI of each bead specificity (represented by a dot in the plot) is highlighted if it is more than three standard deviations outside of the normal range. When the user clicks on a dot, the software displays additional information about the bead including the bead number, MFI, count, mean, standard deviation and the historical mean plus and minus three standard deviations. The software also shows a threshold value of 5000 MFI (each laboratory can set their own threshold) to identify bead IDs where the historical and current values of the positive control fall below a threshold. For each bead specificity, the user can easily compare a box plot of the historical values of the bead's positive control against the value from the current tray. The software shows a Done button that closes the view of the positive control and returns the user to the visualization of the plate. The user can also click a Discard Edit button to postpone review of the current well. Upon return, a marking appears on the positive control well to document that it has been examined in detail.

Once the technician has reviewed the negative and positive control wells, the technician reviews the wells corresponding to patient samples. The software provides specialized detailed views 209 for wells where there was no previous patient sample and wells where prior patient samples are available. When the user clicks on the show detail button in a well for which there was no previous samples available the software shows the following. The software shows a plot with general information about the well including, patient first name, patient last name, patient medical record number, date of the plate, string identifier of the plate, the location of the well in the A-H row, 1-12 column coordinate system, and the catalog ID of the plate. The software also provides the wells internal negative and positive control MFIs and the number of low count beads. This information serves as critical reference points for interpreting the results. Before the user would have to gather this information from multiple sources, but the software presents everything in one coherent visualization. The software provides a text box where the user can enter, edit, and automatically save remarks about their analysis. The software also shows the user a pareto plot of the normalized bead intensities. One dot is plotted for each bead type. Internal positive and negative controls are distinguished by having a larger size. The software enables the user to click on any dot to show the bead number, MFI, and bead count. The user synthesizes the information about the controls and bead plot to determine if the sample is OK or if it needs retesting. The software has a button to mark the sample as OK and another button to mark the sample as requiring retesting 211. When the user clicks the OK or retest button, the user is returned to the plate visualization 205 and the well is either marked with an icon to indicate that the well is OK or needs retesting. This enables the user to easily identify what work is done, what work remains, and not miss any wells that need evaluation.

When the user clicks on the show detail button in a well for which there are previous samples available the software shows the following. The software shows general information for the current sample as well as for the most recent previous sample: patient first name, patient last name, patient medical record number, date of the plate, string identifier of the plate, the location of the well in the A-H, 1-12 coordinate system, and the catalog ID of the plate. The software also provides the current sample well's internal negative and positive control MFIs and the number of low count beads. The software computes similarity metrics to help the user understand if the current and prior samples are similar or significantly different; the metrics include the Sum of the MFI ratios and the # of high MFI ratios. Multiple statistics are shown for each well, each of them is color coded black or red to indicate whether it falls outside its expected range to direct the user attention to possible problem areas, furthermore, in one embodiment, the software uses a decision tree to combine the multiple statistics into a single color code that is used for the background of the cell, to ensure that the user is at a glance aware of problem areas and possible spatial correlations. The software provides a text box where the user can enter, edit, and automatically save remarks about their analysis. The software provides two visualizations to compare the current and previous sample. The first visualization shows a grouped bar chart that, for each bead, shows one bar for the MFI of the current sample, and a second bar for the normalized intensity of the previous sample. This enables the user to quickly spot if the MFI of each bead has markedly changed between the previous and current sample. The second visualization shows a scatter plot of the current and previous MFI bead intensity. The software overlays on the scatter plot a confidence area that helps the user spot beads with MFI that are substantially different between the current and previous patient's sample. The software has a button to mark the sample as OK and another button to mark the sample as requiring retesting. When the user clicks the OK or retest button, the user is returned to the plate visualization and the well is either marked with an icon to indicate that the well is OK or needs retesting.

The software provides a button to indicate that the analysis of all wells on a plate has been completed. When the user clicks the button, the software gathers the results and uses a custom predefined template to generate a PDF report that summarizes the analysis 213. The PDF report includes a header to identify the technician who performed the work, the laboratory name, the address, and the CLIA lab number. The report includes a visualization of the tray annotated with the results, a summary of the controls, a summary of the analysis of each patient, and the comments collected during the analysis. The software shows a Sign button that lets the user digitally sign the report. The software provides digital signature management that fully complies with Part 11 and ensures a fully auditable review trail for every report. The software also has a Send Back button to enable the user to reject the report and provide feedback for additional analysis. The software shows a text box to allow the reviewer to enter comments on the analysis that will be passed on to any additional reviewers or to the prior user if additional work is needed. The software automatically creates pending tasks for the next user in the process which can be the technician that needs to perform rework or another approver that needs to review and process the report 215. All reports are stored in a database to facilitate clinical workflows and laboratory audits. The software enables the user to download at least one report for use outside of the software, if needed.

When a user marks a sample for retesting the software orders the repeat test and flags the current results as questionable for use further analysis. A new process is launched that keeps track of the new sample 211. A list of samples pending retesting at any one time is available. Once a retest is completed in the lab, the software associates the new and old samples and removes the sample from the list of retest work to be completed. The software then detects the candidate retest sample (e.g. a sample belonging to the same patient), the user is asked to confirm whether this indeed corresponds to a retest or is an independent sample. This process enables auditing of how frequently samples need retesting, and how long it takes until they are retested, as well as keeping a trail of multiple tests of the same sample. The software shows the user a side-by-side comparison of the repeated test sample and the questionable sample along with the history of the patient.

The Antibody Quality Check has the advantage of showing all the information needed to review the quality of the entire run and all tests in one view, so the personnel reviewing the tray will easily analyze and approve the run when appropriate. The Antibody Quality Check software, greatly simplifies the analysis and review process, makes it much more accurate than manual analysis and reduces the TAT substantially. The software rapidly flags samples that need repeats and focuses work on samples where analysis is urgently needed by clinicians.

### Unacceptable Antigen Configuration

In one form of the invention, before running the Unacceptable Check, the user may run the Unacceptables Config process to configure the settings for their transplant center. The software can limit the persons that can perform the configuration, for example, to only the laboratory Director. The software enables the user to provide settings for the UNOS API that will enable upload of unacceptable antigens. For each transplant center, the settings include the transplant center code that identifies the transplant center and the transplant center type. The software shows the user a default specificity that is used to automatically identify beads that may indicate unacceptable antigens. The software allows the user to change this default to reflect the local transplant center preferences. The software also allows the user to create a list of at least one transplant type, e.g. Heart, Kidney, Liver, and specify a default override specificity for each transplant type, because each organ program may have different thresholds of tolerance for unacceptable antigens. Moreover, the software allows the user to specify override thresholds for each organ by creating a list of serologies or alleles that each have their own threshold for automatic identification of unacceptable antigens. By enabling the transplant center to create a comprehensive set of rules for automatically identifying potentially unacceptable antigens, the software enables rapid and consistent implementation of best practices that replace potentially dangerous ad-hoc procedures in place today. The UNOS Unacceptables Config process can be run, by an authorized user, at any time to review and/or edit the configuration.

### Unacceptable Antigen Process

In one form of the invention, the Unacceptable Antigen Check begins by authenticating the person that will perform the work by requiring them to enter a username and password. Once the user is logged into the software they are presented with a list of processes that they can perform. The user runs an instance of the Unacceptables Antigen Check process.

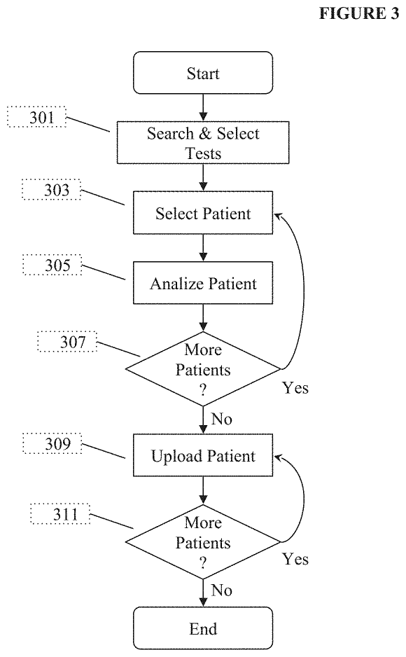

A process flow diagram of the Unacceptable Antigen Check is illustrated in FIG. 3. The first step in the process 301 is to identify plate test results that have recently been completed. The software provides controls to enable the user to search an external database (SQL Server from Fusion, etc.) for tests within the last D days and optionally filter the results by transplant type or a list of at least one medical record number. The software provides a button to trigger the search. If the search fails, the software provides error messages to guide the user in correcting any configuration problems. If the search succeeds, the software provides the user with a table that lists the test results for at least one patient. For each test result, it shows the medical record number, the patient last name, first name, if the test is for SAB1 or SAB2, the date of the sample, if the sample has been analyzed, and any comments written by technicians while preparing the samples and performing the Antibody Quality Check. The UNOS Unacceptable check requires at least one test of type SAB1 and one of SAB2. The software automatically identifies the most recent SAB1 and SAB2 test result for each patient and automatically selects them for further analysis. This saves the technician time by quickly identifying the test results that urgently need attention and minimizing the risk of failing to analyze a test result. The user has the ability to adjust which tests will be analyzed in the current process, in case the default selection is inappropriate. The software provides a Finished button to allow the user to proceed to the next step in the analysis once they are satisfied with the list of tests to analyze.

The second step in the process 303 is to review the list of patients that need analysis for unacceptable antigens and select a patient for analysis. The software provides a progress indicator that shows how many patients are to be analyzed, how many have been analyzed, and how many have had their information uploaded to UNOS. This enables the technician to manage their workflow and understand how much work remains. The software also provides a list of patients by medical record number, last name, first name, and transplant type, along with a status that indicates if the patient requires analysis or upload to UNOS, and a button to start the analysis for each patient, if it has not been done already. When the user clicks the Analyze button for a patient, they are prompted to enter their UNOS username and password to authenticate to the UNOS system. If the authentication fails, the user is presented with a message to help them correct any configuration errors. If the authentication succeeds, the user is taken to the next step in the process to analyze the unacceptable antigens. The software also stores an authentication token associated with the username that will provide continued access to the UNOS API for a fixed amount of time so that the user does not need to enter their username and password every time they need to exchange information with UNOS.

In another embodiment, the Unacceptable Antigen Process can be triggered automatically by completion of an Antibody Quality Check. When an Antibody Quality Check is completed, the software automatically adds an Unacceptable Antigen task to the list of pending tasks to be completed by personnel.

The third step in the process 305 is to analyze the plate bead data for a particular patient to identify the antigens that are unacceptable. The software presents the user with general information about the patient such as the medical record ID, last name, first name, and transplant type. The software also gathers from the database information about the plates that will be analyzed including the SAB1 positive and negative control values, the SAB2 positive and negative control values, and any comments from the technicians that performed the historical Antibody Quality Check. In addition to the general information about the plates and samples, the software presents a table that lists one row for each well/bead along with the antigen serology, molecular alpha, molecular beta, and MFI specificity that was measured during the tests. The user can sort the table on any column. The sort order is customized for each column to provide the most meaningful sort order for serologies, molecular alphas, molecular betas, and MFIs. The user can also filter the table by a particular serology locus such as: A, B, Bw4, Bw6, C, DP, DQ, DR. The software intelligently maintains the sort order of the table as filters are applied and removed to help the user maintain context. The software also automatically highlights MFI specificities that are above the thresholds that are set in the Unacceptable Antigen Config process to help the user consistently identify antigens that are above the thresholds that are set by the transplant center as best-practices. If a threshold is specified for the transplant type, that value is used for the automatica highlighting. If any overrides for a transplant type are specified at the serology or allele level, those overrides are then applied. If no overrides are provided for a transplant type, the global threshold is used for highlighting. The intensity of the color indicates how far beyond the threshold the number is. The user applies a combination of filtering, sorting, and highlighting to identify the unacceptable antigens. The software provides a checkmark box in the table directly next to each serology, molecular alpha, and molecular beta, to allow the user to mark an antigen or allele to be avoided. By co-locating the control for marking the avoided antigen or alpha or beta allele and the MFI for that bead, the risk of erroneously avoiding or allowing an antigen is greatly reduced. The software will automatically ensure that if a serology or alpha or beta allele in one row is checked or unchecked by the user that all other rows with that same specificity are automatically checked or unchecked to match. When the user has selected all specificities, serology, molecular alpha, and/or molecular beta alleles to be avoided, the software gathers the current list of unacceptable antigens and submits them to a UNOS api to compute a score called Computed Panel Reactive Antibodies. (CPRA). The CPRA score is primarily used in transplants involving the kidneys and pancreas as an estimate of the number of donors that are incompatible with the patient. The software also computes the Likelihood of a Compatible Donor (LCD) score. Understanding the tradeoff between marking an antigen as unacceptable and the relative size of the potential donor pool is critical to making sound decisions, especially when unacceptable antigen values are close to the guideline thresholds. Immediate display of the CPRA score enables the user to delicately balance the risk of unacceptable antigens to a potential organ match against the likelihood of not finding a matching organ in time for a critically ill patient. UNOS created a sliding-scale point system that prioritizes patients in the organ allocation algorithm to increase the donor availability to highly sensitized patients with a high CPRA. Therefore, patients with a CPRA over 80% will receive points that will locate them above similar patients without points in the match run. In one extreme, patients with 99% and 100% CPRA, receive the highest points and also are allowed to receive regional and national donor offers respectively. Therefore, providing an accurate and timely upload of Unacceptables for highly sensitized patients is of great importance to maximize the opportunities to transplant a patient. The software provides a button that lets the user conclude the analysis for a patient. When the user clicks the button the software gathers the unacceptable antigens from the table and saves them for the next step. The software also computes two flags, one that indicates that the patient has been tested for unacceptable antigens and one that indicates if at least one antibody specificity had a value greater than zero. These flags are required for uploading the unacceptable antigens to UNOS. In the case that no unacceptable antigens are flagged, but there were some wells with MFI specificities greater than zero, this saves the user from having to remember this fact in the subsequent upload step. The software uses a predefined and configurable mapping to convert the selected antigens from their textual representation as serology, molecular alpha, and molecular beta alleles, to lists of integer indices for each antigen class as required by UNOS. This greatly reduces the likelihood of a clerical error and accelerates the upload process as compared to using a website or having to construct a file manually for upload.

The process has a decision point 307 where software then checks to see if all patients in the patient list have been analyzed, if not, it returns the user to the patient listing page 305 and updates the progress indicators to reflect that one more patient has been analyzed so that the user can pick the next patient to analyze. If all patients have been analyzed, the software advances to a screen 309 that allows the user to review and upload the unacceptable antigens for each patient to UNOS.

Before analyzing a patient 305 or uploading a patient 309, The software examines the saved UNOS authentication token to determine if the token is still valid, if a new token can be obtained via a simple refresh request, or if the user must enter their username and password to authenticate again. The user is prompted, if necessary, to enter their username and password, thereby saving them from entering the same information multiple times within a day.

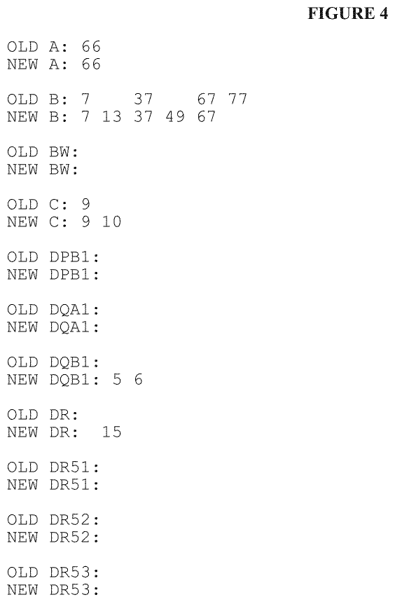

In the upload step in the process 311, the software presents the user with a progress indicator that shows how many patients unacceptable antigens have been uploaded to UNOS as a fraction of the total patients that have been analyzed and remain to be uploaded. The software helps the user work one patient at a time to confirm the unacceptable antigens and upload them to UNOS. For each patient the software does the following. The software shows general information about the patient including their medical record identifier, last name, first name and transplant type. The software allows the user to select the transplant type from a predefined list if the transplant type could not be read from the database. The software also allows the user to enter the patients UNOS Registration Number that is required by the UNOS Upload API. The software has a button that queries UNOS for the currently registered unacceptable antigens. The query result also includes an eTag that is required to upload new antigens. The software automatically stores the eTag for later use in the Upload. The software then displays a unique visualization FIG. 4. that shows the new proposed unacceptable antigens next to the old antigens listed in UNOS so that the user can easily spot changes that are being made for each class of antigen.

The software then provides the opportunity to upload the patients unacceptable antigens to UNOS. When the user clicks the upload button, the software automatically verifies that the software has an authenticated connection to the UNOS system, gathers the eTag that is required for submitted along with the unacceptable antigen lists, and the flags that indicate that the patient has been tested for HLA, and the flag that indicates if any MFI Specificity was greater than zero into the appropriate format for submission to the UNOS API. The software posts the new information to UNOS and receives a response. The software checks the response for errors. If there are errors, they are presented to the user for correction. Otherwise, the software advances to information about the next patient to upload and updates the progress indicator accordingly. The software provides an Exit button that allows the user to leave the process. Normally, the user exits the process after all patients that have been analyzed have been uploaded. The software, however, remembers where the user left off in their process if they leave the process and wishes to pick up where they left off at a later time.

Finally, all data associated with every step in the Unacceptable Antigen check are automatically time-stamped and saved to a server. The software allows users to search completed tasks for the purpose of completing audits. This is a separate capability from that of reports of each instance of a process. A completed task is shown with the same user interface and data as when the task was completed, but it is immutable. Thus in particular it is possible to see what values were being seen by the user and were entered. Completed tasks are searchable through a variety of criteria, including performer user name, process name, task name, completion date and or business key (a semantically meaningful unique identifier associated with each instance of a process that includes key initial values provided to the process instance when it is launched).

The software also provides an interface for reviewing and auditing all UNOS Avoids Check tasks that are ongoing or completed. The software shows the user a list of tasks. For each task, the software shows who performed the task, the name of the process in which the task was performed, the name of the task, and the task completion date and time. The software indicates which tasks are complete and which are still in progress. The user can filter the list on who performed the task, the name of the process, the name of the task, a range of dates, or by keyword search. The software lets the user select a task from the list, and click a button to show the data that was collected from the task using exactly the same interface that the original user used to complete the task. The software even keeps track of prior versions of the tasks, so that if new features or data are added, older tasks in the history can be displayed as they were at the time they were run or completed. This provides an immutable audit trail that enables swift gathering of information for compliance reviews. This is useful, because UNOS requires that the Acceptable antigen uploads and reviews are documented. Also, laboratories inspected by ASHI and CAP may be required to show documentation of this review. The software provides tracking and documentation of the process that is an important part of the quality assurance of the laboratory.

### Virtual Crossmatch Process

The Virtual Crossmatch software is designed to rapidly analyze all the patient's relevant antibody data against a particular donor. When a patient or several patients from the same center received a donor offer, a Virtual Crossmatch is requested to further assess the compatibility of the patient(s)-donor. In one form of the invention, the software downloads the donor typing from UNOS, compares patient and donor HLA typing determining the match grade base in UNOS policies and displays all the antibody information as potential donor specific antibodies. In one view, the reviewer can evaluate all relevant information, from the matching grade, to the MFIs of the potential DSAs by single antigen beads. The reviewer can select what information will be displayed in the final report and submit each patient report for review to the final approver. This software makes the Virtual Crossmatch for several patients a smooth, accurate, timely and well-documented process.

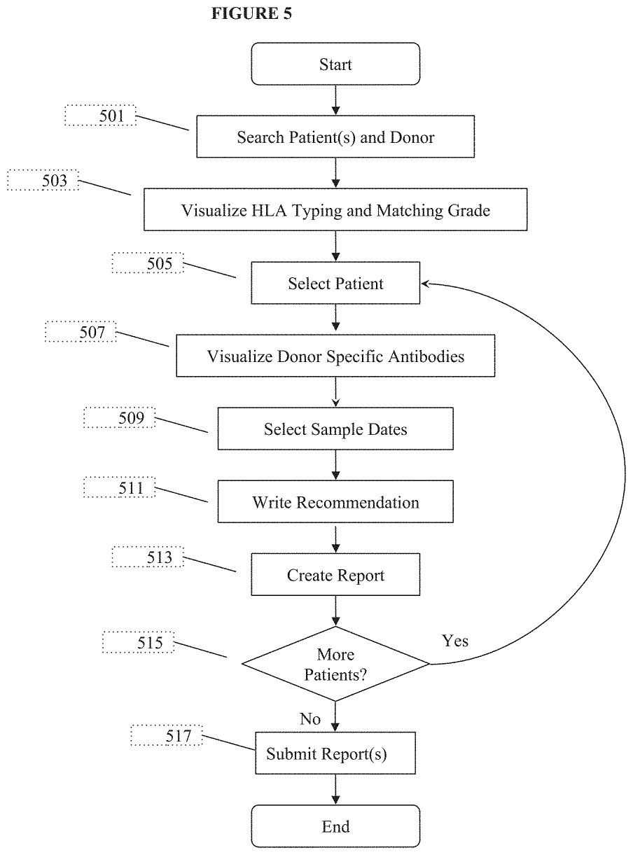

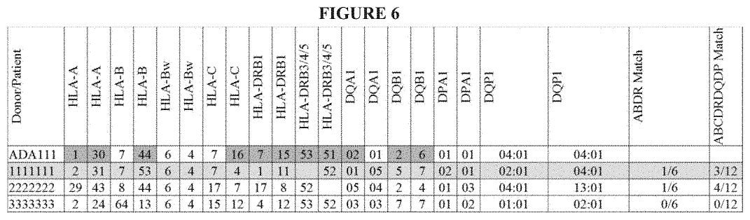

Referring to FIG. 5., more specifically, when UNOS performs a match run for a donor and at least one patient from the center are in the candidate list, the transplant center may request a comprehensive assessment of each patient from the HLA laboratory. This detailed assessment is named Virtual Crossmatch. After the request, the Virtual Crossmatch software is used to do the analysis, review and report the assessment in a well-documented signed report. Initially, the transplant center will request the Virtual Crossmatch assessment for a particular donor and one or several patients by sending to the Match ID, the UNOS donor ID and the names and medical record (MRN) numbers of the patients. The laboratory technician on call, upon receiving the request, will open the Virtual Crossmatch Process software and enter the search for the patient and donor 501 by entering: match run ID, the patients' MRN and the donor ID into the software. In another embodiment, the transplant center may use the software to electronically open the virtual crossmatch request with the HLA lab. Next, the software will help the user authenticate to the UNOS website and retrieve the donor HLA typing from the UNOS website. The software will also gather the patients' HLA typing and antibody data from the laboratory databases. Next 503, the software will display the HLA typing in a tabular representation FIG. 6. with the donor and patient or patients typing and the match grade for HLA-A, B and DR, as well as for all loci. For calculating the matching grade, the software applies a known algorithm that compares the 2 antigens per one locus of the donor with the 2 of the patient for the same locus, and counts how many matches the donor has with the patient. Then, the software does the same count for each locus that matching grade refers to (matching grade for HLA-A, B, DR: 3 loci). In the case of HLA-A, B and DR (3 loci), the software counts the matches for the 3 loci, at a maximum of 2 matches per locus, the maximum matches is 6/6 (six out of six possible matches). If there are no matches at any loci, the match grade for HLA-A, B, DR is 0/6 (or zero out of six possible matches). Similarly, the match grade is calculated for HLA-A, B, C, DR, DQ and DP with a maximum matching grade of 12/12 (twelve out of twelve possible matches). These 2 matching grades are shown in the last two columns of the tabular window FIG. 6.

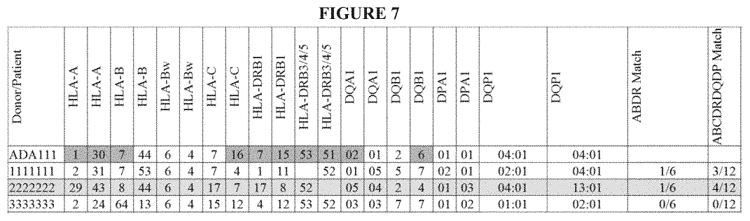

The software let's the user select a patient FIG. 5 505 by clicking the field of one of the patients' MRN, first column in the same tabular window. Next, the software FIG. 5 507 highlights donor mismatches with the selected patient. Screenshots show the interaction in FIG. 6 and FIG. 7.

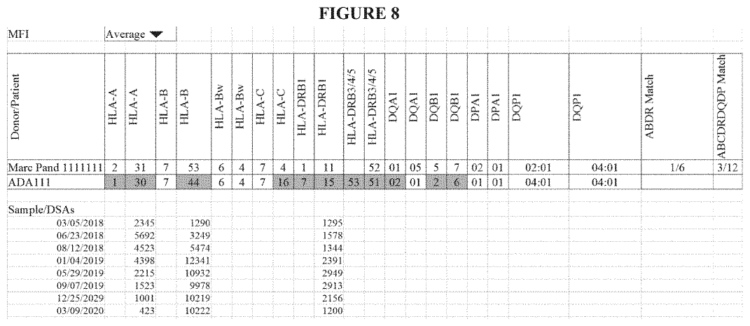

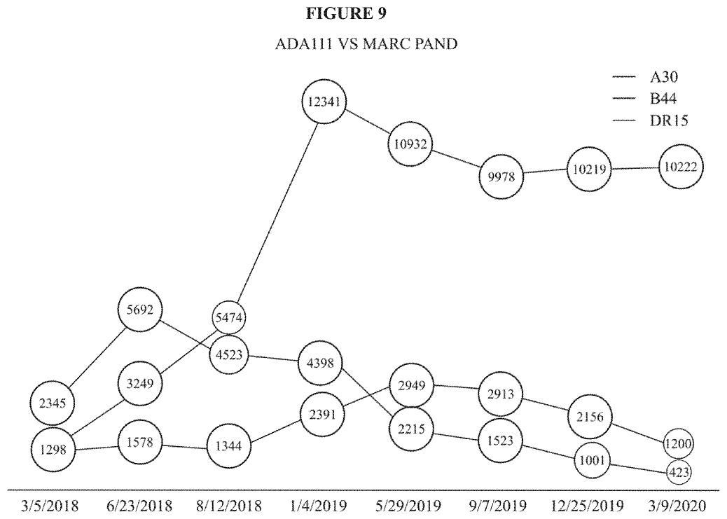

Upon clicking on the patient MRN link (first column in the table), the software will show the patient and donor typing with a list of sera tested for the particular patient and the MFIs for each DSA FIG. 8. The software provides a mechanism to select the MFI calculation when there are more than one bead with same antigen specificity to select the average, range, minimum or maximum of the beads MFIs. The software has a button to trigger display of a plot of the MFI for each DSA against the dates tested FIG. 9. This representation helps the user to understand the history of the DSA. Referring back to FIG. 5, after reviewing the MFI information in the tabular representation, the technologist will select a set of representative sera dates 509, write a recommendation 511, and create a report 513 that will be submitted to the next reviewer. In most of the cases, the last reviewer is the laboratory director or general supervisor on call that will review all the data and add a recommendation to the transplant program about accepting or declining the donor offer. The Virtual Crossmatch may be repeated for multiple patients, 515. When all patients have been analyzed, a final report is submitted 517 to the transplant program with creator and reviewers signature. The report and digital signatures are saved and available for audits. The software provides the HLA lab with an easy, accurate and timely way of doing Virtual Crossmatches. This enables the HLA laboratory to provide swift answers to the transplant program for deciding if they should accept or decline an offered organ.

The foregoing description of a preferred embodiment of the invention has been presented for purposes of illustration and description, and is not intended to be exhaustive or to limit the invention to the precise form disclosed. The description was selected to best explain the principles of the invention and practical application of these principles to enable others skilled in the art to best utilize the invention in various embodiments and various modifications as are suited to the particular use contemplated. It is intended that the scope of the invention not be limited by the specification, but be defined by the claims of the patent.

We claim:

1. A method reducing error rates, managing risk, reducing turnaround time for histocompatibility laboratory processes, and for demonstrating compliance with laboratory standards, using computer software, comprising the steps of:
Displaying a user interface for configuring connections to at least one database, the interface including controls for providing the information to secure a connection to each database;
Keeping a list of laboratory tasks that need to be performed;
Authenticating a person by comparing information provided by the person against known facts;
Identifying which tasks can be performed by an authenticated person based on at least one role assigned to the person and at least one task type assigned to each role;
Displaying a list of tasks that can be performed by a person and enabling the person to select a task to perform;
Removing a task from the list of tasks when a person has completed the task;
Storing the result of a task so that the state of the user interface at task completion can be faithfully reconstructed;
Displaying a user interface for finding and auditing information stored about completed tasks.

2. The method of claim 1, wherein the step of displaying the user interface for configuring database connections includes, for each database, text-entry controls for entering the database URL, the database driver name, the database name, and authentication information selected from the group consisting of: username and password, fingerprint, voice, face recognition, or possession of a trusted objects.

3. The method of claim 1, further comprising summarizing the work performed in a task into a document and saving the document.

4. The method of claim 3, further comprising displaying a user interface for configuring a template for header information that will be included in all documents.

5. The method of claim 4, wherein the step of displaying a user interface for configuring a report header that will be used at the top of all documents, the interface including: a text-entry field for the laboratory director's first name, a text-entry field for the laboratory director's last name, a text-entry field for the name of the laboratory, a text-entry field for the address of the laboratory, a text-entry field for the name of the hospital, a text-entry field for the laboratory's CLIA identification number, a button for saving changes to the template, a button to restore the template to a default state, a button to discard changes made while editing the template.

6. The method of claim 3, further comprising digitally signing documents by a configurable set of users, comprising the steps of:
Determining if a person is authorized to define the signature sequences for each task type;
Displaying a user interface to define, for each task type, a sequence with at least one role that can sign documents generated by that task type;
Displaying a user interface for a person to review and digitally sign a document.

7. The method of claim 1, wherein the step of displaying a user interface for finding and auditing information stored about completed tasks includes replicating the user interface of the task at the moment of task completion, and displaying documents associated with the task.

8. The method of claim 7, wherein the displaying a user interface for auditing tasks step shows a table of completed tasks with columns for the performer of the task, the name of the process from which the task was triggered, the name of the task, the task completion date and time, and the name of the instance of the task, a pull-down menu for filtering the table by performer, a pull-down menu for filtering the table by process, a pull-down menu for filtering the table by task, a date-entry control for excluding tasks before a date, a date-entry control for excluding tasks after a date, a text-entry area for filtering the table by keyword search, a button for running the keyword search, a button to display detailed information about the selected task in the table.

9. A method for reducing error rates, managing risk, and reducing turnaround time for assessing the quality of histocompatibility immunoassay results, using computer software, comprising the steps of:
Identifying when results of an immunoassay plate become available for analysis by periodically checking a database to identify new immunoassay test results completed since the last periodic check;
Creating a task to analyze immunoassay results when new results are identified and adding the task to the list of tasks that need to be performed;
Identifying wells on a plate where information about at least one previous sample for the same patient are available for comparison;
Computing a score that indicates the degree of similarity between the current sample in a well and at least one previous sample, where available;
Displaying a user interface for completing assessment of the quality of an immunoassay plate's results that visually indicates if a well passed or failed quality evaluation;
Displaying a user interface for completing an evaluation of the quality of the sample in a well, including a control to approve the quality of the sample in the well and a control to reject the quality of the sample.

10. The method of claim 9, wherein identifying the availability of a plate is accomplished by Running a cyclic BPMN process on a timer that periodically checks the laboratory database for the plate results and compares the date and time of the results against the date and time of the most recent plate already processed to determine all plates that require processing.

11. The method of claim 9 wherein, the step of computing a score is calculating the sum of the ratio of MFI values for each bead type in the current sample and the immediately prior sample.

12. The method of claim 9 wherein, the step of displaying a user interface for assessing quality of the immunoassay plate shows a grid visualizing the location of each well on the immunoassay plate, where each cell in the grid is color coded to indicate if the corresponding well was empty, contained a control sample, contained a patient sample where bead counts are below a configurable threshold, contained a patient sample for which there is no previous sample, the value of the similarity score for the well's sample and at least one previous sample, showing a checkmark on cells where a person has approved the quality of the sample in the well, showing an “X” mark on cells where a person has rejected the quality of the sample in the well, a button on each cell to launch a task to perform an evaluation of the quality of the sample in the well, and a button to complete the plate assessment task.

13. The method of claim 9 wherein the step of displaying a user interface for assessing quality of the immunoassay plate, color coding is used to indicate if current sample is similar, dissimilar, or very dissimilar based on user configurable score thresholds.

14. The method of claim 9, wherein the step of displaying a user interface for completing an evaluation of the quality of the sample in a well shows a chart comparing the normalized intensity of each bead on the current and prior sample, a button for the person to approve the quality of the sample in the well, a button for the person to reject the quality of the sample in the well and create a task for a person to repeat the sample.

15. The method of claim 9, further comprising identifying when a sample from a well has been approved and then creating a task to analyze antigens.

16. The method of claim 9, further comprising identifying all wells where the quality was rejected and creating tasks for a person to repeat each rejected sample.

17. The method of claim 9, further comprising generating a report document that shows the results of analyzing the wells on a plate and creating at least one signature task for required signature roles and orders, and storing the document.

18. The method of claim 17, further comprising displaying a user interface for reviewing a report document, a control to digitally sign the document, a control to send the task from which the report was generated back into the task list for further analysis.

19. The method of claim 18 wherein the step of displaying a user interface for reviewing a report document shows a window displaying the report, a button to sign the report, a button to send the task from which the report was generated back into the work queue for further analysis, a pull-down-menu to select the person or role to which the further analysis should be assigned, and a text-entry region where the person can document their review.

20. A method for reducing error rates, managing risk, and reducing turnaround time for determining unacceptable antigens and uploading the unacceptable antigens to UNOS, using computer software, comprising the steps of:
Specifying UNOS connection parameters including at least one pair of transplant center code and transplant center type;
Recommending which antibodies should be avoided by selecting a method from the group consisting of: beads where measured values exceed a configurable global threshold, beads where measured values exceed an organ-specific threshold, and beads where measured values exceed the higher of an organ-specific threshold and organ-loci-specific threshold;
Displaying a user interface for exploring statistical information about antibodies, visually indicating if beads exceed threshold rules, showing controls to specify which antibodies to be avoided when matching a donor, showing a comparison of the avoids currently registered with UNOS against the avoids proposed by the person, displaying the CPRA for the selected antibodies, displaying the CPRA for avoids currently in UNOS, a control to upload the antigens to UNOS;
Converting antibodies to be avoided to the required UNOS format, connecting to UNOS via API, and uploading the antibodies to be avoided.

21. The method of claim 20, wherein the step of displaying a user interface for statistical information about antibodies has a text-area showing the catalog IDs used to map numeric bead IDs to human-readable antigen labels, a table with columns indicating serology, molecular alpha, molecular beta, and MFI, one row for each bead from the class I and class II immunoassays, the table sortable by clicking on a column header, the table filterable by locus from a pull-down-menu, the MFI column highlighted when the MFI exceeds threshold rules, a checkbox in each populated cell to enable the person to mark the serology, molecular alpha, or molecular beta to be avoided, a button to automatically select avoids according to configurable thresholds, a button to clear all selected avoids, querying the UNOS API for CPRA of the current and proposed avoids, a text display of the current and proposed display, a comparison of previous and proposed avoids for each locus, a button to upload the proposed avoids to UNOS and save the task results.

22. A method for reducing error rates, managing risk, and reducing turnaround time for performing a virtual crossmatch between a donor and at least one patient, using computer software, comprising the steps of:
Identifying when an organ becomes available and matches at least one patient at a transplant center;
Gathering identifiers for the donor and at least one patient for a virtual crossmatch from the group consisting of: displaying a user interface for entering identifiers for the donor and at least one patient, and retrieving the identifiers from a database;
Gathering donor typing information from the group consisting of: displaying a user interface to upload at least one document containing donor typing information, and retrieving donor typing information from at least one database;
Creating a task to perform a virtual crossmatch analysis for every patient matched to a donor and adding the task to the list of tasks that need to be performed;
Loading the most recent patient typing information from a database for use in the virtual crossmatch analysis;
Displaying a user interface for performing a virtual crossmatch analysis that displays patient and donor typing information, indicates mismatches between patient and donor, provides a longitudinal view of bead data from the current sample and at least one prior sample, provides controls to allow the person to exclude zero or more beads from the analysis, provides a control for selecting an analysis interpretation from a configurable list of analysis interpretations.

23. The method of claim 22, wherein the step of displaying a user interface for comparing the consistency of the information entered shows a table for Class I typing information with loci across the columns and typing information from the UNOS Donor Summary in one row, and the Lab Donor Report in an adjacent row, where the cells are highlighted if the values don't match, a table for Class II typing information with loci across the columns and typing information from the UNOS Donor Summary in one row, and the Lab Donor Report in an adjacent row, where the cells are highlighted if the values don't match, a display area for simultaneously showing the contents of the UNOS Donor Summary and Lab Donor Report documents to facilitate double-checking, a button to complete the consistency check task.

24. The method of claim 22, wherein the step of displaying a user interface for performing a virtual crossmatch analysis shows a table comparing patient and donor types, the table configurable to show only donor specific antibodies, the table cells color coded to indicate mismatches between patient and donor, a second table with one row per previous sample, aligned under the first table showing the a user-selectable statistic such as maximum, minimum, range, or mean WI for all beads associated with the allele in the sample, the cells of the second table color coded according to configurable rules, a third table showing for a selected allele and each historical sample, the MFIs for all beads associated with the allele along with a checkbox allowing the person to exclude a bead from being reported, a pull-down menu with a configurable selection of virtual crossmatch interpretations, a button to complete the virtual crossmatch task.

25. The method of claim 22, further comprising generating a report document that shows the results of a completed virtual crossmatch analysis, creating an ordered list of at least one signature tasks for required signature roles and ordering, and storing the document.

26. The method of claim 22, further comprising displaying a user interface for reviewing at least one document, signing a document using a digital certificate, refusing to sign a document, and creating a task for a person, or person with a particular role, to perform additional work.

27. The method of claim 26, wherein the step of displaying a user interface for reviewing at least one document shows a window for displaying a document, a pull-down list for selecting an additional document, a second window for displaying the additional document, a button for signing a document, a button for creating a task to perform additional work, a pull-down list for selecting a person, or role of persons that should be assigned the task of performing additional work.

28. The method of claim 22, further comprising prescribing that the organ from the donor be transplanted to a specific patient to treat the patient's specific medical condition.
